(19) United States
(12) Patent Application Publication
(10) Pub. No.: US 2011/0003400 A1
(43) Pub. Date: Jan. 6, 2011

(54) METHODS AND SYSTEMS FOR GROUND AND SURFACE WATER SAMPLING AND ANALYSIS

(75) Inventor: Rolf U. Halden, Tempe, AZ (US)

Correspondence Address: EDWARDS ANGELL PALMER & DODGE LLP, P.O. BOX 55874, BOSTON, MA, 02205, US

(73) Assignee: THE JOHNS HOPKINS UNIVERSITY, Baltimore, MD (US)

(21) Appl. No.: 12/918,965

(22) PCT Filed: Feb. 20, 2009
(86) PCT No.: PCT/US09/01076

§ 371 Date: Aug. 23, 2010

Related U.S. Application Data
(60) Provisional application No. 61/066,644, filed on Feb. 22, 2008.

(51) Int. Cl. G01N 33/53; B01L 1/00

(52) U.S. Cl. 436/501; 436/73; 422/430

(57) ABSTRACT

The invention provides devices, methods, and kits for collection of dry samples from fluid samples such as ground or surface water. Devices of the invention include a casing including a water intake zone wherein the casing encloses, a fluid reservoir, a pump, a non-aqueous collection matrix cartridge, and a waste water conduit, wherein the water intake zone, the fluid reservoir, the pump, the non-aqueous collection matrix cartridge, and the waste water conduit are all operably linked in sequence. Methods for using the device of the invention are provided. Kits including the device of the invention or for use with the invention are also provided.

### CROSS REFERENCE TO RELATED APPLICATIONS

The instant application is related to U.S. Provisional Patent Application Ser. No. 61/066,644 filed on Feb. 22, 2008 which is incorporated herein by reference in its entirety.

### STATEMENT OF RIGHTS TO INVENTIONS MADE UNDER FEDERALLY SPONSORED RESEARCH

This work was supported in part by NIH Grant NIEHS-R01 1R01ES015445. The government has certain rights in the invention.

### BACKGROUND

Reliable sampling of ground water and surface water frequently suffers from many limitations, including the collection of discrete water samples rather than time-integrated ones; the generation of large volumes of excess water considered hazardous for which disposal can be difficult and expensive; in groundwater sampling, the collection of depth-discrete water samples not originating from the ascribed location of sampling; and also in groundwater sampling, collecting samples of compromised chemistry due to atmospheric gas intrusion (e.g., dissolved oxygen intrusion) from water cascading down following evacuation of a well during well-purging.

### SUMMARY OF THE INVENTION

The invention provides method for collection of a dry sample by contacting a non-aqueous collection matrix with a ground water sample in situ wherein the sample comprises or is suspected of comprising an analyte that binds the non-aqueous collection matrix.

The methods of the invention can be carried out by providing a device including a casing with a water intake zone enclosing a fluid reservoir, a pump, and a non-aqueous collection matrix cartridge. The water intake zone, the fluid reservoir, the pump, the non-aqueous collection matrix cartridge, and the waste water conduit are all operably linked in sequence. Further, the non-aqueous collection matrix cartridge is operably linked to a waste water conduit. The method includes contacting the water intake zone with a fluid sample suspected of containing at least one analyte for binding to the non-aqueous collection matrix cartridge such that the fluid sample sequentially enters the fluid reservoir, the pump, the non-aqueous collection matrix cartridge, and the waste water conduit, thereby collecting a dry sample.

The invention provides devices in which the casing encloses a plurality of non-aqueous collection matrix cartridges. In some embodiments, the plurality of non-aqueous collection matrix cartridges bind the same analyte. In an alternative embodiment, the plurality of non-aqueous collection matrix cartridges bind a plurality of analytes.

The methods of the invention provide for the use of the device in a ground water well in a saturated aquifer. In an alternative embodiment, the device can be used for the collection of a dry sample from groundwater.

Methods of the invention include the use of the device in a ground water well including a screened interval, and optionally further including one or more inflatable liners either above or below the device. When using inflatable liners in the well, it is preferable that the waste water conduit empties distal to an inflatable liner in the well.

The invention further provides methods for a real time sensor operably linked to a non-aqueous collection matrix cartridge. The real time sensor can be used for collecting data continuously or periodically regarding analyte binding to the non-aqueous collection matrix cartridge. Alternatively, the real time sensor can be used for collecting data episodically, e.g., drop of water table, failure of a connection in the device.

By collection of dry samples, rather than fluid samples, the methods of the invention allow for collection of samples for extended time periods. For example, the water intake zone can be contacted with a fluid sample for about 1 second to about 1 year, for example for about 1 minute, about 1 hour, about 1 day, about 1 week, about one month, about 3 months, about 6 months, about 9 months, or a year.

The method of the invention provides for the performance of an in well purge using a purging a device having a minimum length approximated by a formula:

Volume of water [cm³]=(radius of the well [cm])*(radius of the well [cm])*π*length of the discharge line 607 [cm],

wherein π is ˜3.14, and the radius of the well is half the inner diameter of the monitoring well.

The invention further provides for dry samples prepared by any of the methods of the invention.

The invention further provides devices that can be used for practicing the invention including a casing having a water intake zone wherein the casing encloses, a fluid reservoir, a pump, preferably a multi-channel pump, and one or more non-aqueous collection matrix cartridges wherein the water intake zone, the fluid reservoir, the pump, the non-aqueous collection matrix cartridge, and the waste water conduit are all operably linked in sequence. The non-aqueous collection matrix cartridge is operably linked to a waste water conduit. The device can further include a tether for positioning the device in a collection well. The tether can further be used to operably links the device to a control system.

In some embodiments of the invention, each of the non-aqueous collection matrix cartridges all bind the same analyte. In other embodiments of the invention, the non-aqueous collection matrix cartridges bind a plurality of analytes.

In some embodiments of the invention, the device further includes a real time sensor operably linked to a non-aqueous collection matrix cartridge. The real time sensor can be used for collecting data continuously or periodically regarding analyte binding to the non-aqueous collection matrix cartridge. Alternatively, the real time sensor can be used for collecting data episodically, e.g., drop of water table, failure of a connection in the device.

The invention provides devices optionally including a discharge line having a minimum length approximated by a formula:

Volume of water [cm3]=(radius of the well [cm])*(radius of the well [cm])*π*length of 607 [cm],

wherein π is ˜3.14, and the radius of the well is half the inner diameter of the monitoring well.

The invention further provides kits for practicing the methods of the invention and for use with devices of the invention including instructions for use of a device of the invention and one, two, three or more components for use with the device such as: a casing including a water intake zone, a fluid reservoir, a pump, a non-aqueous collection matrix cartridge, a waste water conduit, and connector tubing. The invention further provides kits including a plurality of non-aqueous collection matrix cartridges for use with the methods or any of the devices of the invention and instructions for use.

### DEFINITIONS

“Analyte” is understood as any compound that may be present in a sample that can be captured using a non-aqueous collection matrix and detected using an assay or method.

By “cartridge” is meant a container enclosing the solid matrix through which the sample is passed through or over. The solid matrix is enclosed in the cartridge to allow the sample to pass through the cartridge, for example into an inlet port and out of an outlet port, wherein the solid matrix is retained within the cartridge.

By “concentration” or “concentration of the analyte” as used herein is understood as decreasing the volume in which a given mass of an analyte is present. For example, decrease the volume in which the given mass analyte is present by at least at least 2-fold, at least 10-fold, at least 10²-fold, at least 10³-fold, at least 10⁴-fold, or at least 10⁵-fold.

“Contacting” as used herein is understood as bringing two components into sufficient proximity (e.g. a groundwater sample containing or potentially containing an analyte and a non-aqueous collection matrix that can bind the analyte, a fluid sample and the water intake zone of the device) for sufficient time and under appropriate condition of temperature, pressure, pH, ionic strength, etc. to allow for the interaction of the two components, e.g., the binding of the analyte to the non-aqueous collection matrix, the entry of water into the device through the water intake zone. Contacting in the context of the invention typically occurs in a non-aqueous collection matrix container such as cartridge, column, or other device that allows the water to flow through the container in a path to allow the water to contact the non-aqueous collection matrix. Contacting a non-aqueous collection matrix cartridge is understood as contacting the matrix within the cartridge with the fluid sample.

“Control system” as used herein is understood as a device such as a computer or recording device. The control system can be used predominantly for mechanical uses, such as positioning the device in the well. The control system can also be used for turning on and off various components of the device, such as the pump, opening and closing fluid lines in the pump, directing collection of a time integrated or time discrete sample, etc. The control system can also be used for the purpose of data collection in the form of electronic data, or by attachment to a chart recording device. The control system can be physically attached to the device by wires or cables. Alternatively, a wireless control system can be used with the device.

As used herein, “detecting”, “detection” and the like are understood as an assay or method performed for identification of a specific analyte in a sample. The amount of analyte detected in the sample can be none (zero) or below the limit of detection (<LOD), positive and within the calibrated range, or positive and outside of the calibrated range of the assay or method.

“Distal” is understood herein as meaning further away than, typically relative to the device of the invention. For example, a waste line that empties distal to an inflatable liner empties on the far side, i.e., the opposite side, of the liner when viewed from the device. The side of the inflatable liner facing the device would be “proximal” to the device.

“Dry sample” as used herein is understood as the non-aqueous collection matrix cartridge after it has made contact with a fluid sample, such as groundwater or surface water, wherein at least one analyte is suspected of or known to be bound to the non-aqueous collection matrix in the cartridge. A dry sample can contain water or other fluid. All moisture does not need to be evacuated from the cartridge. However, the sample contains no more fluid that will fit in the cartridge with the non-aqueous collection matrix present in the cartridge. Both time-discrete samples and time-integrated samples can be converted to dry samples by use of a non-aqueous collection matrix cartridge. Conversion of aqueous to dry samples may occur in the subsurface (i.e., in situ) or on-site prior to shipping of samples.

As used herein, “kits” are understood to contain two or more components of the device of the invention, or components for use with a device of the invention, in appropriate packaging or with instructions for use.

“In situ” as used herein is understood as in the subsurface, preferably at or near the site that the sample is collected. “At or near the site that the sample is collected” is understood as at the same or similar depth such that pressure changes have little or no effect on the sample from the time that the sample is collected to the time that the sample is contacted with the non-aqueous matrix. It is understood that lateral movement within the well will typically have far less effect on pressure in the sample than movement in the depth in the well. In situ contacting of samples with a non-aqueous matrix is differentiated from contacting the non-aqueous matrix with the sample at the surface (i.e., ground level) when the sample is collected in the subsurface. It is understood that contacting surface water with the non-aqueous matrix at the site of collection (i.e., at ground level) is understood as contacting the sample with the matrix in situ.

As used herein, “interchangeable” is understood as the device being designed so that one or more components of the device can be readily exchanged for a similar component. For example, lines and non-aqueous collection matrix cartridges can be joined using bayonet connectors, rapid release connectors, quick connectors, screw connectors, compression connectors, Luer lock, or other similar type connectors that require no tools for the separation or connection of components. Further, non-aqueous collection matrix cartridges can be exchanged depending on the site of groundwater to be tested, the type and quantity of analyte to be detected, and the quantity of water to be tested. Similarly, tubing or other connectors for example from the pump to the non-aqueous collection matrix cartridges may be changed depending on the analyte to be detected to prevent adsorption into the tubing, or the volume or flow rate of the water to be tested. Interchangeable parts such as tubing or cartridges can be disposable. Such considerations are well understood by those of skill in the art.

As used herein, “non-aqueous analyte collection matrix”, “matrix”, “resin”, and the like are understood as material or a mixture of materials that are designed to come into contact with the fluid sample and, through their relatively greater affinity relative to water, will remove and concentrate the analyte or analytes of interest from the fluid sample including dissolved solid, gas, and particulate materials of interest. For example, groundwater or surface water can be passed through, over, or mixed (i.e., contacted) with the non-aqueous analyte collection matrix, thereby causing this matrix to bind and concentrate one or more analytes. It is understood that the binding properties of the materials for one or more specific analytes can depend on various properties of the sampled fluid, for example, ionic strength, pH, etc. The material can bind the analyte(s) specifically, e.g., chelator EDTA for binding heavy metals, peptide metal binding motifs, antibodies for binding desired antigens, molecular pockets formed by molecular imprinting, or specific and nonspecific binding sites relying on van-der-Waals forces, hydrophobic interaction, hydrophilic interaction, mixed-mode interaction, hydrogen bridges, affinity binding sites, etc. Alternatively, the material can bind the analyte(s) based on charge, e.g., cation exchange, anion exchange or mixed-mode ion exchange materials. The analyte collection matrix does not need to be a solid. It can be a non-aqueous liquid, a gel or a semi-solid that attracts and concentrates the analytes by the mechanisms mentioned above as well as by chemical partitioning out of the water and into the analyte collection matrix. The matrix can be contacted with the liquid sample in any known format, including a column, bulk binding, etc. Such methods are well known to those of skill in the art.

“Obtaining” is understood herein as manufacturing, purchasing, or otherwise coming into possession of.

“Operably linked” is understood as a connection, either physical or electronic, between two components of the device, or a component of the device and a remote sensor, data collector, controller, computer, or the like such that the components operate together as desired. For example, a fluid line operably linked to a non-aqueous collection matrix cartridge is understood as a fluid line that delivers fluid to the non-aqueous collection matrix cartridge without loss of fluid and at the desired flow rate. A device operably linked to the controller can be moved to the desired position in the well, and the pump or other components of the device can be turned on or off using the controller.

As used herein, “plurality” is understood to mean more than one. For example, a plurality refers to at least two, three, four, five, ten, 25, 50, 75, 100, or more.

As used herein, “real time” is understood as while the process is occurring, for example, collecting data, and preferably transmitting data to a device or person, at the same time the sample is being collected. The data need not be transmitted instantaneously, but is preferably transmitted within about 1 minute, 2 minutes, 5 minutes, 10 minutes, 15 minutes, or 30 minutes from the time that it was collected, or the collection of the data packet was completed. Data can be sent continuously or periodically in real time for monitoring the progress of a process, or can be sent episodically, e.g., upon overload of a non-aqueous collection matrix cartridge, failure of the device, detection of water table, completion of in well purge, etc.

A “sample” or “fluid sample” as used herein refers to a material, particularly ground water or surface water that is suspected of containing, or known to contain, an analyte. A fluid sample can include dissolved gases, as well as any dissolved or particulate solids. Methods and devices of the invention can be used for the collection of gases as well as dissolved or particulate solids upon selection of the appropriate non-aqueous collection matrix. A reference sample can be a “normal” sample, from a site known to not contain the analyte. A reference sample can also be taken at a “zero time point” prior to contacting the cell with the agent to be tested. A reference sample can also be taken during or after collection of a time integrated sample. A reference sample is typically a time discrete sample when it is collected at the same site as a time integrated sample.

As used herein, “time-discrete sampling” is understood as collection of a sample over a short period of time, for example, less than about an hour, less than about 30 minutes, less than about 15 minutes, less than about 10 minutes, less than about 8 minutes, less than about 5 minutes, less than about 4 minutes, less than about 3 minutes, less than about 2 minutes, less than about 1 minute. Time discrete sampling can also be known as “grab sampling.” The amount of time over which the sample is captured is dependent on a number of considerations known to those of skill in the art. Such considerations include, but are not limited to, the concentration of the analyte in the sample, sensitivity of methods for detection of the analyte in the sample, the maximum yield of the water source samples, the kinetics of binding to the extraction matrix, the temperature of the water sampled, etc.

As used herein, “time-integrated sampling” is understood as the collection of one or more samples, wherein each sample is collected over an extended period of time, for example at least about 1 hours, at least about 2 hours, at least about 3 hours, at least about 4 hours, at least about 5 hours, at least about 8 hours, at least about 12 hours, at least about 15 hours, at least about 18 hours, at least about 21 hours, at least about 24 hours, at least about 36 hours, at least about 2 days, at least about 3 days, at least about 4 days, at least about 5 days, at least about 6 days, at least about one week, at least about 8 days, at least about 9 days, at least about 10 days, at least about 2 weeks, at least about one month, at least about 3 months, at least about 6 months, at least about 9 months, at least about one year, or more. The amount of time over which the sample is captured is dependent upon a number of considerations well known to those of skill in the art. Such considerations include, but are not limited to, the concentration of the analyte in the sample, sensitivity of methods for detection of the analyte in the sample, tidal changes or other changes that would affect the height of the water table through the collection time or the direction of water flow and thus the composition of the water sampled, capacity of binding non-aqueous collection matricies for analyte mass, kinetics of mass transfer to the extraction material, etc.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

Any devices or methods provided herein can be combined with one or more of any of the other devices and methods provided herein.

### BRIEF DESCRIPTION OF THE FIGURES

FIG. 1 shows a cross-section of a subsurface showing the deployment of the sampling device in a groundwater monitoring well;

FIG. 2 is a schematic of a preferred embodiment of the sampling device and system showing its various components;

FIGS. 3A and 3B are schematics illustrating the collection of time integrated samples in a groundwater monitoring well and concomitant concentration of various analytes in multiple sampling collectors;

FIGS. 4A and 4B are schematics of a preferred embodiment of the analyte collector showing the concentration of a specific analyte from groundwater over time;

FIGS. 5A and 5B are schematics of a preferred embodiment of the analyte collector equipped with a real-time sensor suitable for in situ detection of analytes concentrated from groundwater;

FIGS. 6A and 6B are schematics of in-well purging using a multi-channel, variable-speed pump in combination with two inflatable liners to prevent discharged groundwater from reentering the sampling zone. Purged water may be discharged in the screened zone (A) or into the well casing (B); and

FIG. 7 is a schematic of in-well purging using a multi-channel, variable-speed pump in combination with a long discharge line several feet in length to transport purged groundwater away from the sampling zone.

### DETAILED DESCRIPTION AND PREFERRED EMBODIMENTS

The devices and methods provided herein offer significant benefits over currently available techniques for groundwater and surface water collection including, but not limited to, (i) generating time integrated samples; (ii) limiting the amount of waste generated; (iii) allowing for a standardization of sample information collected by traditional methods and the methods provided herein; (iv) allowing for the collection of multiple samples preserved for analysis of individual analytes of interest; (v) improving the limit of detection for analytes of interest; (vi) allowing for the preservation of liable sample constituents in situ; (vii) concentrating and compartmentalizing analytes of interest in situ, and (viii) through the concentration and compartmentalization step, allowing for the detection of analytes in situ through real time sensing technology.

The time interval between collection of the sample and testing of the sample can alter the sample in traditional water collection methods. For example, as the sample is brought from the site of collection in the subsurface to the ground surface, the pressure changes. This changes the solubility of gases such as carbon dioxide, and thus potentially the pH of the water. Changes in pH are coupled to changes in the solubility of the analytes which therefore may affect the accuracy and precision of the analysis. Intrusion of gases, particularly oxygen, is an important limitation of traditional samples and collection methods. Oxygen may enter into the volume of water sampled during pumping, during the lift to the ground surface, at the ground surface, during shipment of the sample, or during handling in the analytical lab. Although the sample can be packaged in a way to expel all of the air in the container, oxygen and other dissolved gases are typically present in the sample which can react with various analytes in the sample. Further, although the sample can be degassed, this can also result in changes in the analytes present in the sample.

Binding of the analyte(s) to a solid matrix using the devices and methods of the instant invention stabilize the analyte(s). As a substantial portion of the water has been removed from the analyte, effects of moving the sample are substantially minimized, if not removed. This allows for improved integrity of the sample(s) resulting in greater accuracy and precision in sample analysis. Methods of the invention further include the use of preservatives and/or reactants to stabilize certain analytes or generate stable reaction products. Such preservatives can be liquid or gas, or a combination thereof. Selection of such a preservative or reactant would be dependent on the analyte to be preserved. Appropriate preservatives and reactants can be selected by those of skill in the art.

The advantages provided by the devices and methods of the invention include: (i) the use of a submersible multi-channel pump in conjunction with non-aqueous collection matricies, solid phase extraction cartridges, and similar devices to concentrate the analyte(s) of interest in the solid phase, thereby eliminating the need for retrieval, transport, analysis, and disposal of large amounts of water during monitoring activities; (ii) use of a submersible multi-channel pump in conjunction with inflatable liners for in-line purging without generation of purge water above the surface; (iii) use of in situ analyte concentration in conjunction with real time sensing technology; and (iv) analysis arrays allowing for the determination of concentrations of multiple analytes in discrete channels using analyte-specific preservation techniques, concentration steps, and sensing technology.

The devices, systems, and methods provided herein may be used to compare to or replace traditional grab sampling (time-discrete sampling) with time-integrated sampling, thereby eliminating uncertainty as to the occurrence and impact of temporal water quality changes. This type of sampling is especially valuable in sampling locations that are under the influence of tidal water movement. The concentration and/or capture of analytes on solid matricies eliminates the need for costly transport and disposal of water samples. The functionality of concentrating analytes over time in an analyte collector results in improved detection limits, allowing for the detection of lower concentrations of analyte(s) in original samples by concentration of analyte(s) using solid matricies. The use of solid matricies to capture analyte(s) permits in situ testing and/or sensing of analyte(s) in a sample without removing the sampling device from the site of sample collection.

Presently available devices and methods for sample collection are limited by the amount of water that can be stored and transported from the source and to a laboratory for analysis. Currently available devices can collect about 1 L to about 4 L samples.

The apparatuses, systems, and methods of the invention provide for the collection of “dry samples.” In other words, the analytes of interest contained in a volume of water predetermined by the flow rate set at the multi-channel pump, rather than the size of the fluid container, are transferred from the dissolved in the fluid phase to the sorbed phase or simply trapped in a non-aqueous collection matrix. Transfer of the analyte from the water onto a solid non-aqueous collection matrix eliminates the need to retrieve the water itself. As a result, there is no need to ship large amounts of liquids, which is a costly undertaking and difficult given the new restrictions of transport of liquids on airplanes and other carriers. The collection of “dry samples” reduces analysis costs and eliminates the need of disposing of large volumes of liquid hazardous waste as the fluid is not removed from the original site.

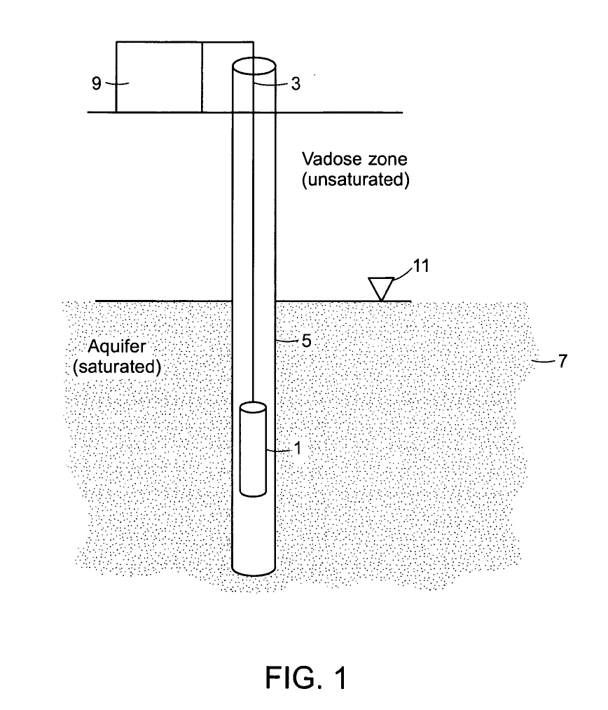

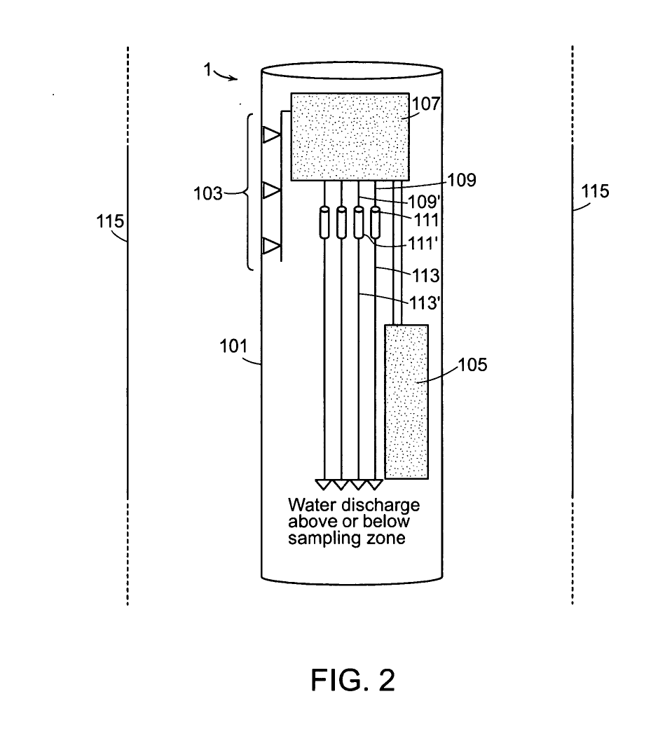

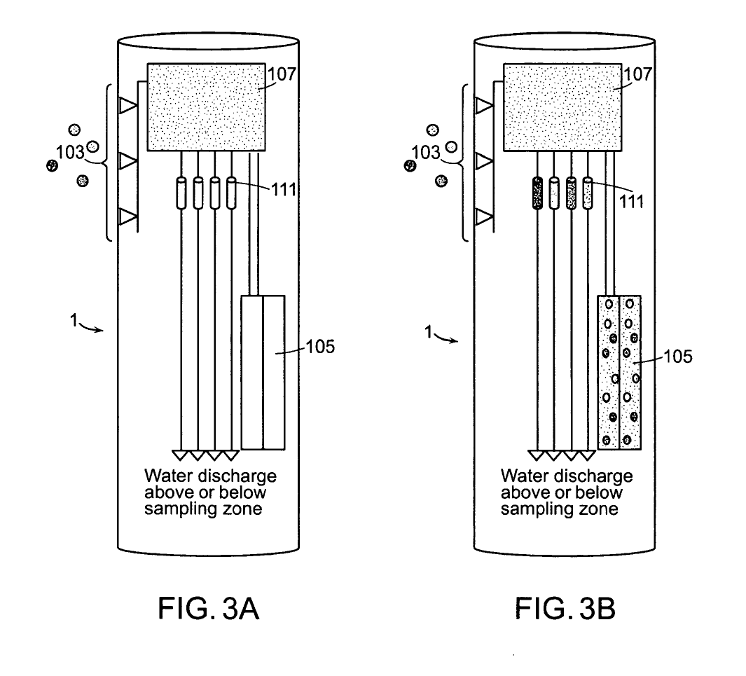

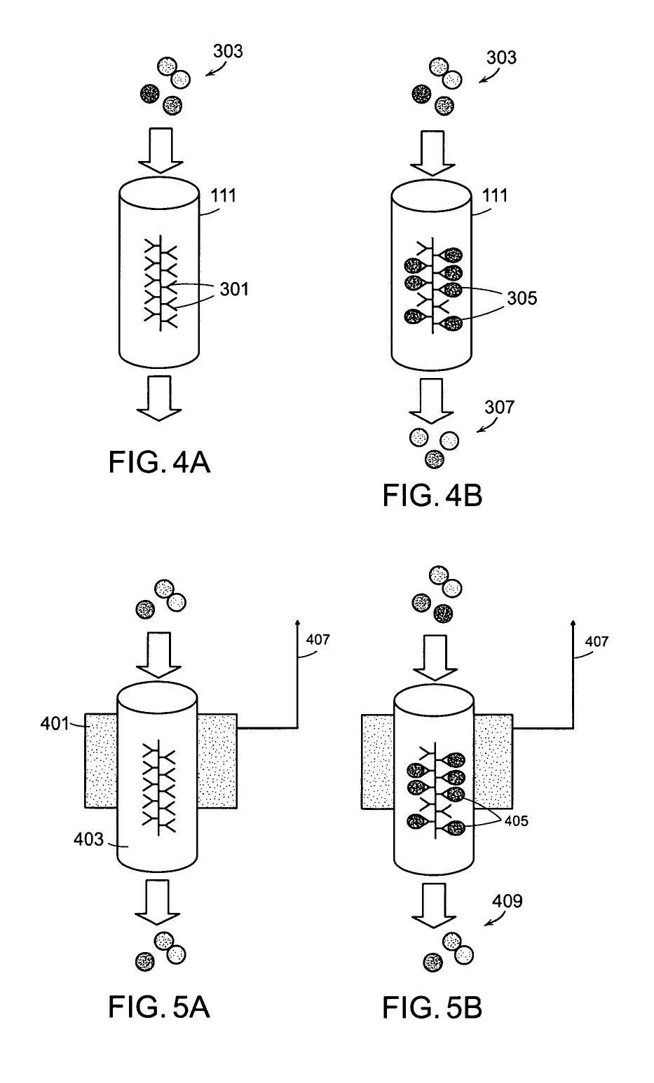

Briefly, the schematic in FIG. 1 shows the deployment of the sampling device in a groundwater well. The device includes a multi-channel pump and various analyte collectors. Upon deployment of the device in the screened interval of a given groundwater monitoring well, the multi-channel pump is turned on. The pump then delivers a known and pre-set volume of water per unit time through inert tubing to an array of analyte collectors. The analyte collectors, shown in FIG. 2, consist of a capsule or module containing a medium suitable for trapping a specific analyte of interest, e.g., lead (Pb), chromium (Cr), or organic contaminants of concern. Selective trapping of the analyte(s) of interest simultaneously in multiple parallel channels, shown in FIG. 3, is achieved using techniques including solid phase extraction (SPE), ion exchange resins, affinity chromatography resins, molecular imprinting resins, etc., shown in FIG. 4. If desired, the pre-concentrated analytes can be assayed in situ using real-time sensing technology, as shown in FIG. 5.

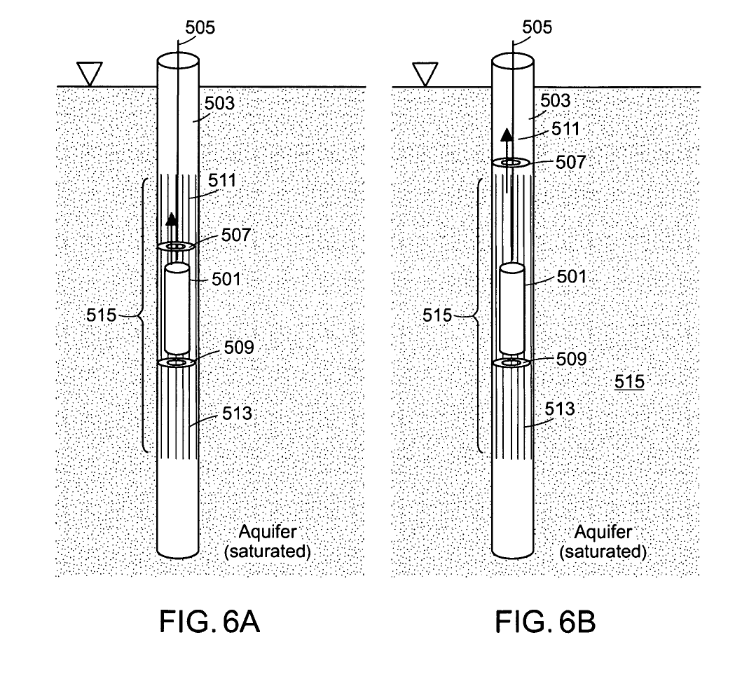

Water is delivered by the pump for a period sufficient to contact between 10 mL and greater than 100 L of water with the analyte collector. Sampling periods may vary from a few seconds to days, weeks or several months. Resultant flow rates thus can vary from one or more liters per minute to 1 milliliter per day. Filtration material placed between the water intake and the analyte collector can remove microorganisms to prevent in situ degradation of analytes of interest. Water that has passed through the sample collector is discharged either above or below the sampling zone as shown in FIG. 3. Inflatable liners may be used to prevent water that has passed through the device from reentering the sampling zone as shown in FIG. 6.

Similarly, the inflatable liners also may be used to isolate the sampling zone during high flow in-well purging. This in-well purging process offers great utility. First, it prevents the generation at the surface of large volumes of water that have to be disposed of as hazardous waste. Second, by isolating a region in the well from the upper atmosphere via the use of the inflatable liner, atmospheric gases are precluded from entering the sampling zone. This prevents the accidental oxygenation of water which is known to cause artifacts in chemical analysis of redox sensitive species. Also, since the well casing does not need to be completely evacuated, as is often the case in wells of low productivity, water cannot cascade down in the screened interval which again would result the potential reoxygenation, outgassing, and other sampling artifacts having detrimental effects on the water chemistry and analysis. During in-well purging, unwanted pure water also can be discharged far away from the sampling zone via use of a discharge line in lieu of, or in combination with, inflatable liners such as those shown in FIG. 6. Upon termination of the sampling period, the device is retrieved and the loaded cartridge or cartridge containing the non-aqueous collection matrix suitable for trapping a specific analyte of interest can be shipped to laboratories for analysis.

FIG. 1 is a schematic of a device of the invention deployed for the collection of groundwater. The device 1 attached to a tether 3 is lowered into a groundwater monitoring well 5 drilled into an aquifer 7. The tether is appropriately attached to the device so that the device will be appropriately oriented in the well. The opposite end of the tether 3 is attached to a structure 9 on the surface to retain the device 1, wherein the structure can include a control system. When attached to a control system, the tether typically includes wires, cables such as fiber optic cables, or other components to transmit information from the device to the control system. In an alternative embodiment, the tether is predominantly or purely structural to retain the device at the desired position and information from the device is transmitted wirelessly to a control system either at or near the well, or at a remote location. The groundwater level is indicated by the downward pointing arrowhead 11.

FIG. 2 is a schematic of a device 1 of the invention. The device is enclosed in a casing 101 which includes a water intake zone 103 operably linked to a reservoir, preferably a multi-compartment reservoir 105 for collection of groundwater between the water intake zone and a pump. Such a reservoir can be useful for the collection of a time discrete sample before, after, or during the time interval sample collection by segregating the time discrete sample from the time integrated sample, or for partitioning the time integrated sample into multiple identical wells to allow the sample to be drawn into one or more channels of the pump 107. The pump is preferably a multi-channel pump. Each of the one or more channels of the pump 109 and 109′ is operably joined to a first end of a corresponding non-aqueous collection matrix cartridge 111 and 111′. The specific size and identity of the non-aqueous collection matrix in each of the cartridges depends on the type and amount of sample to be collected, the binding capacity of the non-aqueous collection matrix, and other considerations well known to those of skill in the art. The amount of extraction matrix may vary from 1 mg to 10 or even 100 grams based on the specific application; this range of sorbent materials allows sampling of water volumes ranging from 1 ml to 1 L to hundreds or even thousands of liters. In a preferred embodiment, duplicate or triplicate cartridges of the same non-aqueous collection matrix are present in the device to act as controls. Alternatively, or additionally, non-aqueous collection matricies that bind the same analyte based on different binding interactions can also be used as a control. The second end of the corresponding non-aqueous collection matrix cartridge if operably attached to a conduit 113 and 113′ such as a rigid tubing to discharge the water above or below the sampling zone after being passed through the non-aqueous collection matrix cartridge. A screen extending beyond the height of the device is represented by 115. One or more filters or membranes can be incorporated into the device to prevent clogging of the non-aqueous collection matrix cartridge, e.g., between the pump and the cartridge to provide sufficient pressure to push the sample through the membrane. Such filters and membranes are well known to those of skill in the art.

FIGS. 3A and B provide a schematic of the device of the invention in use. Groundwater enters the device 1 through the water intake zone 103. Water is collected in the multi-channel reservoir 105 for the desired time period, either short or long, and then the pump 107 is used to apply the sample to the non-aqueous collection matrix cartridges 111 at the appropriate flow rate. The separation process is demonstrated schematically. The reservoir includes spots of four different shades of gray. After passing through the pump and contacting the non-aqueous collection matrix columns 111, each of the columns has turned the same the same shade of gray as one of the spots, representing that each column binds a specific analyte. As in the previous figure, the water from the columns is discharged either above of below the sampling zone to prevent contamination of the sample. It is understood that some types of non-aqueous collection matricies can bind more than one analyte.

FIGS. 4A and B provide a schematic of an individual non-aqueous collection matrix column 111 that binds a single analyte. Prior to exposure to the sample, the non-aqueous collection matrix includes multiple empty analyte binding sites 301. After contacting the non-aqueous collection matrix with the sample 303, the analyte 305 that specifically binds the non-aqueous collection matrix is bound to the non-aqueous collection matrix in the column. The analytes or other components of the sample that do not bind the non-aqueous collection matrix 307, passes through the column without binding to the non-aqueous collection matrix.

FIGS. 5A and 5B show an embodiment of the invention wherein a real time sensor 401 is attached to the non-aqueous collection matrix column 403 to allow for detection of the analyte 405 bound to the column. In the embodiment, the real time sensor is further connected, with 407 or without wires, to a data logger to record the presence of the analyte bound to the sensor. Data can be sent to the data logger at timed intervals, continuously, upon a certain event such as saturation of the column. In an embodiment, a real time sensor can be used to analyze the flow through 409 that does not bind to the column.

FIGS. 6A and 6B provide schematics of the device 501 of the invention deployed in well 503 for the collection of groundwater. The device 501 is attached to a tether 505 that can be used to lower the device into the well and adjust the height of the device in the well. The tether can also include wires, cables, or other transmission devices to allow for communication between the device and the surface. Upper 507 and lower 509 inflatable liners are in the well above and below the device, respectively. Water is discharged from the device into the well distal to the liners either above the upper liner 511 or below the lower liner 513. The liners prevent discharged water from reentering the well at the sampling site, but do not prevent transport of groundwater into the well from the sides of the well. The well can further include a screened interval 515 for the removal of particulates from the groundwater to prevent clogging of the pump and/or the columns. The inflatable liners can be present in the well in the screened interval or outside of the screened interval. Alternatively, screens can be included in the water intake zone of the device. Appropriate screens and their methods of use are well known to those of skill in the art.

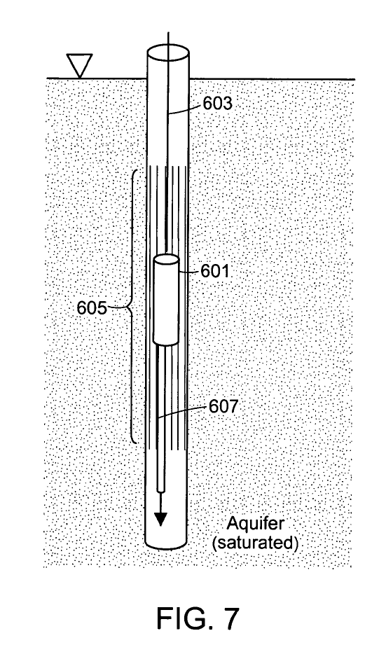

FIG. 7 provides a schematic of an in well purging using the pump in the device of the invention 601 attached to a tether 603 in a well with a screened interval 605. The device is operably connected to a long discharge line in the well 607 that extends beyond the length of the screened interval 605 in the embodiment shown. In an embodiment, one or more inflatable liners can be present in the well (not shown). The required length of the discharge line 607 is a function of the inner diameter of the borehole, the volume of water sampled and the retention time of water in that zone. The convective flow of water through the well screen and bore hole replaces the water in the well to be collected by the device. If the void volume between the discharge point and the pump intake is less than the water volume sampled, there will potentially be short-circuiting and repeated sampling of the same water. This is particularly true if the water is stagnant and does not get replaced through natural convective flow. The volume of the water column between the end of the discharge line 607 and the water intake can be calculated using the following formula:

Volume of water collected [cm³]=(radius of the well [cm])*(radius of the well [cm])*π*length of 607 [cm],

Where it is ˜3.14, and the radius of the well is half the inner diameter of the monitoring well. The specific minimum length of the discharge line to be used can be determined by one of skill in the art depending on the specific circumstances of the well in which collection takes place.

The use of a long discharge line can abrogate the need for the use of one or more liners in the well. However, it is possible to use the device of the invention with a long discharge line in conjunction with one or more liners in the well. In an alternative embodiment, the discharge line is sufficiently long to allow for discharge of the water distal to the liner.

Although the figures and specific embodiments provided herein are related to methods for use with groundwater, the device can also be used for the collection of surface water samples with the appropriate modifications. For example, when samples are collected from open water, the use of liners or other devices to partition off the water to be tested can be difficult if not impossible to use. However, the device of the invention can be used, for example, to obtain samples in moving water, e.g., river or stream, or a body of water adjacent to a river or stream, wherein the water is discharged from the device sufficiently downstream to prevent contamination of the sampling area. In an embodiment, the sample after passing through the device of the invention could be collected in a water tight container and subsequently returned to the sample source. Such modifications are well within the ability of those of skill in the art.

The components of the device, particularly the connector fluid lines between the intake zone, the reservoir, the pump, and the cartridges, as well as after the cartridges to remove waste, are easily interchangeable. For example, the connector fluid lines can be connected to the various components using bayonet connectors, rapid release connectors, quick connectors, screw connectors, compression connectors, luer locks or other connectors such that one or more of the fluid lines can be easily replaced or exchanged depending on, for example, the flow rate through the fluid lines or the analytes to be detected in the sample. For example, some analytes may stick to or corrode specific types of fluid connectors, i.e., plastic tubing. The selection of the specific material for the fluid lines of the device is a matter of choice and within the ability of one of skill in the art.

Further, the specific non-aqueous collection matricies in the cartridges and the volume of the cartridges will depend on the analytes to be collected and the volume of water to be tested. The end user will also consider the number of cartridges to be used in the device at a single time. For example, if a small number of specific analytes are to be detected, e.g., after a known chemical spill, a few larger capacity non-aqueous collection matrix cartridges may be preferable for use in the device and methods of the invention as they typically provide a higher flow rate, shorter collection times, and a higher binding capacity. However, if a new site is being assessed that may have multiple known and unknown contaminants, it may be advantageous for a large number of smaller capacity non-aqueous collection matrix cartridges to be used to allow for the detection of a broader selection of analytes. Such considerations are well understood by those of skill in the art. The specific number and types of non-aqueous collection matrix cartridges used in the device and methods of the invention is not a limitation of the invention.

In certain applications of the invention, it is necessary to employ paired columns as the detection of two analytes is required to determine the level of each. For example, nitrate and nitrite levels must be detected in the same sample. Similarly, the determination of the total concentration of metals may be desirable by tracking in parallel the relative concentrations of different redox species. For example, the total concentration of dissolved iron can be determined by measuring FeII and Fe III in parallel, or the concentration of dissolved uranium can be determined by measuring UN and UVI in parallel.

The embodiments shown in the figures each have one non-aqueous collection matrix column attached to each fluid line from the pump. In an alternative embodiment, more than one cartridge can be attached to a single fluid line from the pump in series as long as contacting the sample with the first non-aqueous collection matrix does not alter binding of the second analyte to the second and possibly subsequent non-aqueous collection matrix(s), and the connection of the cartridges in tandem in a single line does not alter the flow rate through the cartridges. Such considerations are well understood by those of skill in the art.

The invention further provides kits including a device of the invention, or components of a device of the invention. For example, a kit can include the casing with a water intake zone, a reservoir, and a multi-channel pump. The kit can optionally include one or more types of tubing with appropriate connectors for use in the devices or methods of the invention. The kit can include one or more types of non-aqueous collection matrix cartridges, optionally with appropriate tubing and connectors, for use in the devices and methods of the invention. A kit of the invention can include one or more types of non-aqueous collection matrix cartridges appropriately sized and/or with appropriate connectors for use in the devices or methods of the invention. Such a kit would be useful to an owner of the device. Other variations are well within the ability of those of skill in the art.

### Example

### Validation of Collection of Time Integrated Samples of Groundwater

The device and methods of the invention provide a new paradigm for sample collection. The methods of the invention need to be validated and compared to data obtained from traditional collection methods to allow for interpretation of data going forward using time integrated samples in comparison to time discrete samples previously collected. For the purpose of validation, some channels of the multi-channel pump can be directed towards sampling bladders (see FIG. 2) that may be equipped with preserving chemicals (e.g., acids, bases). Captured in these bladders may be water collected slowly over time, e.g., for several days, or drawn quickly at a single discrete time, preferably at the beginning and/or the end of the sampling period, by use of a high-flow rate delivered by the multi-channel pump. All three sampling strategies can be used at a single site to provide the following samples:

1. A traditional water sample, drawn using a multi-channel pump at the beginning of the sample period by the use of a high-flow rate delivered by the multi-channel pump;

2. A traditional water sample, drawn using a multi-channel pump at the end of the sample period by the use of a high-flow rate delivered by the multi-channel pump;

3. A time-integrated water sample, drawn using a multi-channel pump over the entire sampling period by using a low-flow rate delivered by the multi-channel pump; and

4. A dry sample of a specific analyte collected over time in an analyte collector, using the flow rate described in #3.

A comparison of the results obtained from the analysis of the four samples can demonstrate the impact of discrete vs. time-integrated sampling (comparison of 1, 2, and 3), and an evaluation of the analyte concentration efficiency offered by the “dry sample” (comparison of 3 and 4). Similarly, one can test and validate the use of various sample preservation strategies, with the ideal result being that use of a specific preservative that produces indistinguishable results for samples derived by the methods of 3 and 4. Upon validation of the methods, shipment of dry samples will take place in lieu of shipment of fluid samples for laboratory analysis.

Those skilled in the art will recognize, or be able to ascertain using no more than routine experimentation, many equivalents to the specific embodiments of the invention described herein. Such equivalents are intended to be encompassed by the following claims.

All references, patents, and patent publications cited herein are incorporated herein by reference

1. A method of collection of a dry sample comprising contacting a non-aqueous collection matrix with a ground water sample in situ wherein the sample comprises or is suspected of comprising an analyte that binds the non-aqueous collection matrix.

2. The method of claim 1, further comprising:
providing a device comprising:
a casing comprising a water intake zone wherein the casing encloses,
a pump, and
a non-aqueous collection matrix cartridge,
wherein the non-aqueous collection matrix cartridge is operably linked to a waste water conduit, and
wherein the water intake zone, the pump, the non-aqueous collection matrix cartridge, and the waste water conduit are all operably linked in sequence;
contacting the water intake zone with a fluid sample such that the fluid sample sequentially enters the pump, the non-aqueous collection matrix cartridge, and the waste water conduit wherein the fluid sample is suspected of containing at least one analyte for binding to the non-aqueous collection matrix cartridge thereby collecting a dry sample.

3. The method of claim 2, wherein the non-aqueous collection matrix cartridge comprises a plurality of non-aqueous collection matrix cartridges.

4. The method of claim 3, wherein each of the plurality of non-aqueous collection matrix cartridges bind the same analyte.

5. The method of claim 3, wherein the plurality of non-aqueous collection matrix cartridges bind a plurality of analytes.

6. The method of claim 2, wherein the device is present in a ground water well in a saturated aquifer.

7. The method of claim 5, wherein the ground water well comprises a screened interval.

8. The method of claim 6, wherein the ground water well comprises an inflatable liner either above or below the device.

9. (canceled)

10. The method of claim 8, wherein the waste water conduit empties distal to the inflatable liner in the well.

11. The method of claim 2, wherein the device further comprises a real time sensor operably linked to a non-aqueous collection matrix cartridge.

12. The method of claim 11, further comprising collecting data regarding analyte binding from a real time sensor operably linked to the non-aqueous collection matrix cartridge.

13. The method of claim 2, wherein the water intake zone is contacted with a fluid sample for about 1 second to about 1 year.

14. The method of claim 13, wherein the water intake zone is contacted with a fluid sample for about 1 minute, about 1 hour, about 1 day, about 1 week, about one month, about 3 months, about 6 months.

15. The method of claim 2, further comprising an in well purge comprising purging a device further comprising a length approximated by a formula:
Volume of water [cm³]=(radius of the well [cm])*(radius of the well [cm])*π*length of discharge line 607 [cm],
wherein π is ˜3.14, and the radius of the well is half the inner diameter of the monitoring well.

16. A dry sample prepared by a method of claim 1.

17. A device comprising:
a casing comprising a water intake zone wherein the casing encloses,
a pump, and
a non-aqueous collection matrix cartridge,
wherein the non-aqueous collection matrix cartridge is operably linked to a waste water conduit, and
wherein the water intake zone, the fluid reservoir, the pump, the non-aqueous collection matrix cartridge, and the waste water conduit are all operably linked in sequence.

18. The device of claim 17, further comprising a tether.

19. The device of claim 18, wherein the tether operably links the device to a control system.

20. The device of claim 17, wherein the pump is a multi-channel pump.

21. The device of claim 17, wherein the non-aqueous collection matrix cartridge comprises a plurality of non-aqueous collection matrix cartridges.

22. The device of claim 21, wherein each of the plurality of non-aqueous collection matrix cartridges all bind the same analyte.

23. The device of claim 21, wherein the plurality of non-aqueous collection matrix cartridges bind a plurality of analytes.

24. The device of claim 17, wherein the device further comprises a real time sensor operably linked to a non-aqueous collection matrix cartridge.

25. The device of claim 17, wherein the device further comprises a discharge line comprising a length approximated by a formula:
Volume of water [cm³]=(radius of the well [cm])*(radius of the well [cm])*π*length of discharge line 607 [cm],
wherein π is ˜3.14, and the radius of the well is half the inner diameter of the monitoring well.

26. A kit comprising instructions for use of a device of claim 18 and one or more components selected from the group consisting of
a casing comprising a water intake zone, a pump, a non-aqueous collection matrix cartridge, a waste water conduit, and connector tubing.

27. The kit of claim 26, comprising two or more of the components listed.

28. The kit of claim 26, comprising three or more of the components listed.

29. A kit comprising a plurality of non-aqueous collection matrix cartridges for use with the method of claim 1 and instructions for use.

30. The method of claim 2 further including a fluid reservoir operably linked to the pump.

31. The device of claim 17, further comprising a fluid reservoir.

32. The kit of claim 26, further comprising a fluid reservoir.
